(19) United States
(12) Patent Application Publication
(10) Pub. No.: US 2010/0329995 A1
(43) Pub. Date: Dec. 30, 2010

(54) COMPOSITIONS CONTAINING LACTOFERRIN, AND METHODS OF USING SAME TO PROMOTE GROWTH OF SKIN CELLS

(75) Inventors: Scott E. Deeter, Fort Collins, CO (US); Delia R. Bethell, West Sacramento, CA (US); Brandy Sargent, Diamond Springs, CA (US)

Correspondence Address: ARENT FOX LLP, 1050 CONNECTICUT AVENUE, N.W., SUITE 400, WASHINGTON, DC, 20036, US

(73) Assignee: VENTRIA BIOSCIENCE, Sacramento, CA (US)

(21) Appl. No.: 12/280,234

(22) PCT Filed: Feb. 21, 2007
(86) PCT No.: PCT/US07/04388

§ 371 Date: Sep. 14, 2010

Related U.S. Application Data
(60) Provisional application No. 60/774,617, filed on Feb. 21, 2006.

(51) Int. Cl. A61K 38/47; C07K 14/79; C07K 14/415; A61K 9/12; A61P 17/00; A61P 17/02; A61P 17/10; A61P 31/00; A61P 31/04; A61P 31/12; A61P 29/00; C12N 5/071; C12N 9/24

(52) U.S. Cl. 424/45; 530/400; 530/370; 530/377; 424/94.61; 435/325; 435/200; 435/395

(57) ABSTRACT

The present invention relates, generally, to compositions including lactoferrin, and to compositions including both lactoferrin and lysozyme. The present invention also includes topical formulations containing the compositions, methods of making the formulations, methods of using the formulations to treat various skin disorders/conditions, to treat wounds, and to methods of producing artificial skin using the composition, and methods of treating burns using the formulation, optionally in conjunction with the application of artificial skin.

### RELATED APPLICATION DATA

This application claims priority from U.S. Provisional Application No. 60/774,617, which was filed on Feb. 21, 2006, the contents of which are incorporated herein by reference.

### BACKGROUND OF THE INVENTION

1. Field of the Invention

The present invention relates, generally, to compositions comprising lactoferrin, and to compositions comprising both lactoferrin and lysozyme, where the compositions promote proliferation of skin cells, preferably keratinocytes. The present invention also includes topical formulations made from the compositions, methods of making such formulations, and methods of using the formulation to promote skin cell growth, and preferably proliferation and migration of keratinocytes. Such compositions and methods may be beneficially used to treat various skin disorders and wounds. Further envisioned are artificial skin formed using the compositions, and methods of treating burns using one or more of the compositions and the application of artificial skin.

2. Description of the Related Art

The skin is the largest organ of human body, and it is exposed to an ever-changing environment. The primary function of the skin is to serve as a barrier to protect the body against various assaults, such as chemicals, mechanical forces, ultraviolet radiation, and bacterial and viral pathogens. Loss of the integrity of the skin as a result of injury may lead to life-threatening consequences.

The skin is comprised of two major parts: the dermis and the overlying epidermis separated by a sheet-like structure of basement membrane. The dermis mainly consists of a dense collagen-rich matrix connective tissue that provides nourishment and support to the epidermis and provides skin pliability, elasticity, and tensile strength. Fibroblasts are the primary cell type in the dermis and are responsible for the synthesis of the matrix components. The epidermis is made primarily of keratinocytes (approximately 90-95%) that form a stratified squamous epithelium.

Wound healing is a dynamic process consisting of a complex interaction of cellular and biochemical factors. Right after an injury occurs, an inflammatory response phase occurs where platelets adhere and aggregate to the wound site to form a clot, and then inflammatory cells emigrate into and clean up the wound and release cytokines and growth factors. The proliferative phase, where new tissue forms in the wound site, begins within hours after injury with the migration of keratinocytes into wound defect. New stroma of fibroblasts, collagen matrices and new blood vessels begins to form approximately 3-4 days later. The last phase of wound healing involves tissue remodeling to restore normal tissue structure and function, and can last from a few weeks to months or years. The healing process of the proliferative phase involves the migration of keratinocytes from the free edges or residual epithelial structure of a wound, and the proliferation and differentiation (stratification) of keratinocytes to restore an intact epidermis. At the same time, fibroblasts in the wound edges migrate into the wound, and later, produce new collagens and other matrices to repair the wounded dermis. Without the migration and proliferation of keratinocytes and fibroblasts, wound healing would be impossible.

Studies have shown that keratinocyte migration and proliferation are two independent processes; migrating cells do not proliferate. (Sarret, Y., et al., Human keratinocyte locomotion: the effect of selected cytokines. J Invest Dermatol 98: 12-6 (1992).) When migration ceases (possibly due to contact inhibition) keratinocytes re-attach themselves to the underlying substratum, then reconstitute the basement membrane that connects the newly-formed epidermis to regenerated dermis, and resume the process of terminal differentiation to generate a new, stratified epidermis. The mechanism for the keratinocyte migration which initiates the re-epithelialization remains unclear. Studies have found that transforming growth factor beta 1 (TGF-β1) had strong effects on keratinocyte migration (Roberts, A B, and Sporn, M B, Physiological actions and clinical applications of transforming growth factor-beta (TGF-beta). Growth Factors 8: 1-9 (1993)), TGFβ demonstrated stimulate effects on keratinocyte proliferation (Cribbs, R K, et al., Endogenous production of heparin-binding EGF-like growth factor during murine partial-thickness burn wound healing. J Burn Care Rehabil 23: 116-25 (2002)), while epidermal growth factor (EGF) exhibited strong effects on both keratinocyte migration and proliferation (Sutherland, J, et al., Motogenic substrata and chemokinetic growth factors for human skin cells. J Anat 207: 67-78 (2005)).

Due to their key roles in wound healing, efforts have been made to find factors which can promote keratinocyte and fibroblast growth, migration, and differentiation so as to enhance wound healing. Burn wounds treated with recombinant human EGF showed an acceleration of healing by 1 to 1.5 days. (Brown, G L, et al., Enhancement of wound healing by topical treatment with epidermal growth factor. N Engl J Med 321: 76-9 (1989).) In another study, basic FGF was used in randomized placebo-controlled study in 600 patients with second degree burns; the burns treated with basic FGF healed 2 to 4 days faster than burns without basic FGF treatment. (Fu, X, et al., Randomised placebo-controlled trial of use of topical recombinant bovine basic fibroblast growth factor for second-degree burns. Lancet 352: 1661-4 (1998).) The positive effects of EGF, FGF and TGF on wound healing reported in many studies are encouraging. (Review by Fu, X, et al., Engineered growth factors and cutaneous wound healing: success and possible questions in the past 10 years. Wound Repair Regen 13: 122-30 (2005)) However not all studies report the same positive results. One study has shown that treatment with EGF provided no improvement in wound healing. (Cohen, I K, et al., Topical application of epidermal growth factor onto partial-thickness wounds in human volunteers does not enhance reepithelialization. Plast Reconstr Surg 96: 251-4 (1995).) This difference might be due to the complexity of the wound healing process, patient condition, and application time and dose of the growth factors.

Whereas acute wounds go through the wound healing phases outlined above, healing-impaired chronic wounds do not progress through the orderly process. Some areas of the wound are found in different phases, having lost the ideal synchrony of events that leads to rapid healing. More importantly, some cells in chronic wound are phenotypically altered. (Loot, M A, et al., Fibroblasts derived from chronic diabetic ulcers differ in their response to stimulation with EGF, IGF-I, bFGF and PDGF-AB compared to controls. Eur J Cell Biol 81: 153-60 (2002).) Keratinocytes on the edge of chronic wounds are unable to migrate properly and therefore the wound cannot be closed. (Arnold, I, and Watt, F M, c-Myc activation in transgenic mouse epidermis results in mobilization of stem cells and differentiation of their progeny. Curr Biol 11: 558-68 (2001); Stojadinovic, 0, et al., Molecular pathogenesis of chronic wounds: the role of beta-catenin and c-myc in the inhibition of epithelialization and wound healing. Am J Pathol 167: 59-69 (2005); Waikel, R L, et al., Deregulated expression of c-Myc depletes epidermal stem cells. Nat Genet. 28: 165-8 (2001).) One reason for the inability of non-healing keratinocytes to migrate is because they are, for some reason, unresponsive to activation signals that promote cell migration. Fibroblasts of diabetic ulcers showed a decreased proliferative response to TGFβ1 and other growth factors (Hasan, A., et al., Dermal fibroblasts from venous ulcers are unresponsive to the action of transforming growth factor-beta 1. J Dermatol Sci 16: 59-66 (1997); Loot, M A, et al., Fibroblasts derived from chronic diabetic ulcers differ in their response to stimulation with EGF, IGF-I, bFGF and PDGF-AB compared to controls. Eur J Cell Biol 81: 153-60 (2002)), and decreased expression of TGFβ receptor and impaired signal transduction (Kim B C, et al., Fibroblasts from chronic wounds show altered TGF-beta-signaling and decreased TGF-beta Type II receptor expression. J Cell Physiol 195: 331-6 (2003)). Although much more work is needed to clearly define the phenotypical abnormalities in the cells of chronic wounds, these findings have important implications for therapeutic intervention. For example, current topically applied growth factors such as EGF or TGFβ might not find a responsive target cell population in chronic wounds. An agent that has growth factor-like effects on cells and has its own receptors would be a beneficial therapeutic for these chronic wounds.

Another problem with chronic wound sites is that they are often infected. Infection is the most common complication for wounds, and can impair proper healing. Biofilm formation may also occur, leading to increased difficulty in treating the infection, as biofilms are often highly resistant to antibiotics. Biofilms are responsible for diseases such as otitis media, the most common acute ear infection in children in the U.S. Other diseases in which biofilms play a role include bacterial endocarditis (infection of the inner surface of the heart and its valves), cystic fibrosis (a chronic disorder resulting in increased susceptibility to serious lung infections), and Legionnaire's disease (an acute respiratory infection resulting from the aspiration of clumps of Legionnella biofilms detached from air and water heating/cooling and distribution systems). Biofilms may also be responsible for a wide variety of “nosocomial” (hospital-acquired) infections. Sources of such biofilm-related infections can include the surfaces of catheters, medical implants, wound dressings, or other types of medical devices. Very high and/or long-term doses of antibiotics are often required to eradicate biofilm-related infections.

None of the currently available growth factors such as EGF and FGF have antimicrobial effects. It has been shown that microbial counts at a wound site are increased or remain about the same when high concentrations of basic FGF are used. (Hayward, P, et al., Fibroblast growth factor reserves the bacterial retardation of wound contraction. Am J Surg 163: 288-93 (1992).) It would be desirable to implement an agent for wound healing which can promote cell growth and migration and have synergistic effects with EGF, FGF, and other growth factors involved in wound healing. As explained below, LF appears to be the agent.

Lactoferrin (LF) has the potential to be highly effective in aiding wound repair. LF is an iron-binding glycoprotein to which a variety of functions have been ascribed. Not only has LF been shown to have antibacterial and antiviral effects, but also anti-inflammatory properties, which is thought to be mediated in part by the inhibition of TNF-α synthesis. One study showed that topical treatment with LF inhibited IL-1β-induced epidermal TNF-α production on suction blistered skin. (Cumberbatch, M., et al., Regulation of epidermal Langerhans cell migration by lactoferrin. Immunology 100: 21-8 (2000).) LF has also been shown to promote growth of various cell types including bone cells, crypt cells, and epithelial cells of the intestine and the skin.

Lysozyme (LZ), a protein present in human milk, saliva, pancreatic juice, and leukocytes, has also been shown to have bactericidal activity. It has the capability of destroying the cell walls of bacteria by catalyzing hydrolysis of β-1,4 linkages of N-acetylmuramic acid and 2-acetylamino-2-deoxy-D-glucose residues. Therefore, LZ can participate in the defense against bacterial colonization on the skin surface.

U.S. Published Application No. 2003/0190303 describes the use of LF for a potential therapeutic (oral, topical, inhalant, intradermal) to regulate and treat allergy-induced TNF-alpha production as a result of a local immune response from inflammatory skin reactions (acne, psoriasis, contact dermatitis, sunburn, infant diaper rash), asthma, sinusitis, rhinitis, bronchitis, and arthritis. Also, LF can be added to anti-wrinkle cosmetic products to eliminate inflammation caused by hydroxyacids.

U.S. Published Application No. 2004/0214750 describes the use of LF alone or in combination with fatty acids for treating dermatological conditions. The compounds have potential use in preventing wrinkles, and treating acne and rosacea. The compounds have both anti-microbial and anti-inflammatory mechanisms of action.

Published PCT Application No, WO 2005/016372 describes the use of LF as a poly-metal chelator (anti-oxidant), anti-inflammatory (for skin inflammatory disorders), immunostimulatory, and anti-microbial agent for chronic infections in cosmeceuticals, nutriceuticals, and functional foods.

U.S. Published Application No. 2004/0142037 describes the use of rhLF (Aspergillus niger) for treating wounds alone or in combination with other wound healing therapies by stimulating and/or inhibiting certain cytokines and/or chemokines and stimulating cells involved in wound repair. Although the production of keratinocytes as a result of IL-18 or G/M-CSF release from cytokines is disclosed, there is no discussion of directly stimulating production of keratinocytes using LF itself.

Published POT Application No. WO 2001/89540 describes the use of egg whites and a fraction thereof (LZ) in wound healing, including tissue regeneration by increasing cell migration, cell proliferation, prevention of wound infection (anti-microbial, anti-inflammatory), and prevention of scar formation.

Yang et al., “Expression and Localization of Human Lysozyme in the Endosperm of Transgenic Rice” (2003) Planta, 216(4): 597-603 describes expression in rice of human lysozyme under the control of rice regulatory sequences.

Likewise, Hwang et al., “Analysis of the Rice Endosperm-Specific Globulin Promoter in Transformed Rice Cells” (2002) Plant Cell Report 20: 842-847 describes expression of heterologous proteins in rice plants under control of rice regulatory sequences.

None of the above-mentioned techniques addresses the use of LF, or a combination of LF and LZ, to aid in treating various skin disorders and conditions, including, but not limited to acne, aging, age spots, sunburns, inflammation, rosacea, psoriasis, eczematous dermatitis (such as allergic contact dermatitis, atopic dermatitis, nummular eczematous dermatitis, seborrheic dermatitis, vesicular palmoplantar eczema), bites, stings, and infestations. They also fail to address the treatment of skin lesions, disorders, and infections caused by Gram-negative and Gram-positive bacteria, mycobacteria, fungi, yeast, and/or viruses, which may be attributable to, for example, sexually transmitted diseases. Likewise, the above-mentioned techniques do not address the use of LF, or a combination of LF and LZ, for innovative topical preparations for the treatment of wounds, including surgical wounds, where the wounds may be acute or chronic in nature. They also fail to address the use of LF, or a combination of LF and LZ, for the production of artificial skin, and for use in the treatment of burns.

There is clearly a particularly great need in the art for compositions and methods for speeding the healing of wounds. In the US alone, $2.8 billion is spent annually to treat chronic wounds and the worldwide market reaches $7 billion. It has been estimated that 6 million people in the United States were affected by chronic wounds, such as venous ulcers, in 1998, a figure which is most likely underestimated in 2005 in view of the increasing elderly population. In addition, foot ulcers have been shown to occur in 15% of all patients with diabetes and ultimately 84% of these ulcers result in lower-leg amputations. The Centers for Disease Control reports that the prevalence of diabetes has more than doubled in the US from 1998 to 2004 (5.8 million to 14.7 million), and people aged 65 and older now account for 40% of the population with diabetes. Therefore, it is possible that the incidence of chronic wounds associated with diabetes will escalate in future years. The associated morbidity, decreased quality of life, and the high costs associated with the care of chronic wounds have an enormous impact on the economy. Not only are frequent visits required to physicians and nurses, but also loss of productivity, increased frailty in the elderly, requirement of bulky dressings that often need changing, and hospitalization due to potentially life-threatening bacterial infections that may result from the management of slow-healing, chronic wounds. Further, as of Nov. 5, 2005, 15,568 US soldiers involved in Operation Iraqi Freedom were wounded, and about 50% of the injured were unable to return to duty within 72 hours of injury. These acute and chronic wounds can not only threaten the life of the wounded, but can also cause an enormous economic impact on the family of wounded and to the nation as a whole. All of these problems could be partially alleviated by increasing the speed of wound healing.

Accordingly, there is a great need in the art for compositions including LF, or a combination of LF and LZ, for use in promoting skin cell growth (particularly keratinocyte proliferation and migration), for use in the treatment of various skin disorders and conditions, and for use in promoting the healing of wounds. Also needed are topical formulations incorporating said compositions, and methods for making same. In addition, methods of treating skin disorders, conditions, and wounds using the formulations are also needed. There is further a great need in the art for improved artificial skin, and methods for making the same, for use in the treatment of burns, as well as methods of treating burns using one or both of the formulations of the present invention and artificial skin, in order to hasten healing and minimize scarring.

### SUMMARY OF THE INVENTION

The present invention relates to the use of compositions containing lactoferrin (LF) to treat skin disorders and conditions, including, but not limited to, acne, aging, age spots, sunburns, inflammation, rosacea, psoriasis, eczematous dermatitis (such as allergic contact dermatitis, atopic dermatitis, nummular eczematous dermatitis, seborrheic dermatitis, vesicular palmoplantar eczema), bites, stings, and infestations; to treat skin lesions, disorders, and infections caused by Gram-negative and Gram-positive bacteria, mycobacteria, fungi, yeast, and/or viruses, which may be attributable to, for example, sexually transmitted diseases; to treat wounds, including surgical wounds, which may be acute or chronic in nature; and to treat burns and produce artificial skin for use in treating burns. Preferably, the LF is provided as a topical formulation that is prepared in accordance with the methods of the present invention, where said topical formulation is useful for treating skin disorders and conditions, wounds, and burns according to the methods of treatment of the present invention. According to a particularly preferred embodiment of the present invention, the LF topical formulation also includes LZ.

Accordingly, it is an objective of the invention to provide compositions for directly promoting proliferation of keratinocytes, including an effective amount of lactoferrin. Such compositions are useful in treating various skin disorders/conditions and wounds, and in forming artificial skin.

One aspect of the invention comprises a topical formulation for use in directly promoting proliferation of skin cells, including an effective amount of lactoferrin. According to one embodiment, the skin cells being proliferated include keratinocytes, fibroblasts, and skin stem cells. According to a preferred embodiment, the skin cells being proliferated are keratinocytes. According to another embodiment, the topical formulation further includes lysozyme.

Another aspect of the invention comprises a method of promoting keratinocyte proliferation, including the step of contacting keratinocytes with an amount of lactoferrin effective to promote keratinocyte proliferation.

An additional aspect of the invention comprises a method of treating a skin disorder/condition, including the step of applying a formulation containing a therapeutically effective amount of lactoferrin. According to one embodiment, the skin disorder/condition may be any of acne, aging, age spots, sunburns, inflammation, rosacea, psoriasis, eczematous dermatitis (such as allergic contact dermatitis, atopic dermatitis, nummular eczematous dermatitis, seborrheic dermatitis, vesicular palmoplantar eczema), bites, stings, and infestations. According to another embodiment, the skin disorder/condition may be lesions and/or infections caused by Gram-negative and Gram-positive bacteria, mycobacteria, fungi, yeast, and/or viruses, which may be attributable to, for example, sexually transmitted diseases. According to a further embodiment, the method induces proliferation of skin cells, where the skin cells being proliferated are selected from keratinocytes, fibroblasts, and skin stem cells.

Another aspect of the invention comprises a method of promoting wound healing, including the step of applying a formulation containing a therapeutically effective amount of lactoferrin. According to a further embodiment, the method induces proliferation of skin cells, where the skin cells are selected from keratinocytes, fibroblasts, and skin stem cells.

An additional aspect of the invention includes a method of producing a topical formulation for use in wound treatment, comprising the step of providing a therapeutically effective amount of lactoferrin in a pharmaceutically acceptable topical carrier. According to another embodiment, the method further includes providing a therapeutically effective amount of lysozyme.

A further aspect of the invention includes an artificial skin composition for use in treating burns, where the artificial skin is formed by a process of applying lactoferrin to an artificial skin substrate to induce proliferation of skin cells. According to another embodiment, the process further includes application of lysozyme.

Another aspect of the invention comprises a method of treating burns, including the step of applying one or more treatments selected from the group consisting of a topical formulation containing a therapeutically effective amount of lactoferrin, and an artificial skin substance. According to a further embodiment, the method induces proliferation of keratinocytes.

### BRIEF DESCRIPTION OF THE DRAWINGS

Exemplary embodiments of the invention will now be described with reference to the accompanying drawings, in which:

FIG. 1 shows a Coomassie-stained gel analysis of total soluble protein extracted from transgenic rice grain expressing rhLF. Lane 1 represents total soluble protein extracted from non-transgenic rice grain. Lanes 2 to 4 represent total soluble protein extracted from transgenic rice grain of line LF164. Lane 5 represents native human LF obtained from Sigma. The arrow points to the band having molecular weight similar to native human lactoferrin.

FIG. 2 is a graph depicting the pH-dependent iron release from nhLF (native human lactoferrin) and rhLF. Native hLF and rhLF were incubated with 2 M excess ferric iron (FeCl₃:NTA=1:4) and sodium bicarbonate (Fe:HCO³⁻=1:1) for 2 h at room temperature. Excess free iron was removed by using a PD-10 desalting column (Amersham Pharmacia, Piscataway, N.J.) and the iron saturation level was determined by the A₂₈₀/A₄₆₅ ratio. Both nhLF and rhLF were completely saturated with iron. Holo-hLF was incubated in buffers with various pH between 2 and 7.4 at room temperature for 24 h. Iron released from holo-hLF was removed and the iron saturation level was determined by the A₂₈₀/A₄₆₅ ratio.

FIG. 3 compares E. coli colony formation in media with and without 1 mg/ml rhLF, showing reduction of colonies when treated with rhLF.

FIG. 4 depicts the inhibition of bacterial cell growth by LF as measured by optical density at wavelength (A630), Three treatments are control (media only), native (native human lactoferrin) and recombinant (recombinant human lactoferrin).

FIG. 5 is a graph depicting the effect of rhLF on HT29 cell growth. The cell line was grown in baseline media supplemented with 1 mg/ml of rhLF with iron saturation at <10% (apo-LF), about 50% (asis-LF), and >90% (holo-LF).

FIG. 6 is a graph comparing the proliferative responses in HT29 cells when exposed to varying doses of the three forms of LF (asis-, apo-, holo-) as assessed by [³H]-thymidine incorporation.

FIG. 7 is a graph depicting the effect of holo-rhLF on keratinocyte growth. Normal human skin keratinocytes were grown in EpiLife base medium with human keratinocyte growth supplements (HKGS) without transferrin, but with holo-rhLF added at concentrations of 0 (LF₀), 10 (LF₁₀), and 100 (LF₁₀₀) μg/ml. Medium with complete HKGS with transferrin was used as a positive control. 2×10⁴/well cells were plated in 12-well plates at day 0, and cells were cultured for a total of 7 days with cell counting conducted in each day. Each treatment was conducted in triplicate. The Y-axis shows the cell number counts, the X-axis shows the days of treatment, and each bar represents the mean value±the standard error. (See also Chen et al., “Lactoferrin promotes human skin keratinocyte proliferation,” presented on May 2, 2006 at the 19^{th} Annual Symposium on Advanced Wound Care.)

FIG. 8 shows the results of a migration assay in a monolayer of HT29 cells. The monolayer was wounded by scraping a disposable pipette tip across the dish, and the monolayer was then washed with fresh serum-free medium and cultured in serum-free medium in the presence of LF. The rate of movement of the anterior edges of the wounded monolayers was then determined by comparing serial photomicrographs at various times (t=0 h and t=24 h) after wounding.

FIG. 9 compares E. coli colony formation in media with and without 20 μg/ml rhLZ, showing reduction of colony in treatment with rhLZ.

FIG. 10 depicts the inhibition of bacterial cell growth by LZ, as measured by counting colony forming units of E. coli from three treatments: buffer only (black line with white square box), buffer plus native human lysozyme (red line), and buffer plus recombinant human lysozyme (green line).

The invention is also described in more detail below.

### DETAILED DESCRIPTION OF THE INVENTION

Unless otherwise indicated, all terms used herein have the meanings given below or are generally consistent with same meaning that the terms have to those skilled in the art of the present invention. Practitioners are particularly directed to Sambrook et al. (1989) Molecular Cloning: A Laboratory Manual (Second Edition), Cold Spring Harbor Press, Plainview, N.Y., Ausubel F M et al. (1993) Current Protocols in Molecular Biology, John Wiley & Sons, New York, N.Y., and Gelvin and Schilperoot, eds. (1997) Plant Molecular Biology Manual, Kluwer Academic Publishers, The Netherlands for definitions and terms of the art.

General and specific techniques for producing proteins from plant cells may be obtained from the following applications, each of which is incorporated herein in its entirety by reference: U.S. patent application Ser. No. 09/847,232 (“Plant Transcription Factors and Enhanced Gene Expression”); U.S. patent application Ser. No. 10/077,381 (“Expression of Human Milk Proteins in Transgenic Plants”); U.S. patent application Ser. No. 10/411,395 (“Human Blood Proteins Expressed in Monocot Seeds”); U.S. patent application Ser. No. 10/639,779 (“Production of Human Growth Factors in Monocot Seeds”); U.S. Pat. Appl. Ser, No. 10/639,781 (“Method of Making an Anti-infective Composition for Treating Oral Infections”); and international application no. PCT/US2004/041083 (“High-level Expression of Fusion Polypeptides in Plant Seeds Utilizing Seed-Storage Proteins as Fusion Carriers”).

The nucleic acids of the invention may be in the form of RNA or in the form of DNA, and include messenger RNA, synthetic RNA and DNA, cDNA, and genomic DNA. The DNA may be double-stranded or single-stranded, and if single-stranded may be the coding strand or the non-coding (anti-sense, complementary) strand.

By “host cell” is meant a cell containing a vector and supporting the replication and/or transcription and/or expression of the heterologous nucleic acid sequence. Preferably, according to the invention, the host cell is a plant cell. Other host cells may be used as secondary hosts, including bacterial, yeast, insect, amphibian or mammalian cells, to move DNA to a desired plant host cell.

A “plant cell” refers to any cell derived from a plant, including undifferentiated tissue (e.g., callus) as well as plant seeds, pollen, propagules, embryos, suspension cultures, meristematic regions, leaves, roots, shoots, gametophytes, sporophytes and microspores.

The term “mature plant” refers to a fully differentiated plant.

The term “seed” refers to all seed components, including, for example, the coleoptile and leaves, radicle and coleorhiza, scutulum, starchy endosperm, aleurone layer, pericarp and/or testa, either during seed maturation and seed germination. In the context of the present invention, the term “seed” and “grain” is used interchangeably.

The term “seed product” includes, but is not limited to, seed fractions such as de-hulled whole seed, flour (seed that has been de-hulled by milling and ground into a powder) a seed extract, preferably a protein extract (where the protein fraction of the flour has been separated from the carbohydrate fraction), malt (including malt extract or malt syrup) and/or a purified protein fraction derived from the transgenic grain.

The term “biological activity” refers to any biological activity typically attributed to that protein by those skilled in the art.

“Seed components” refers to carbohydrate, protein, and lipid components extractable from seeds, typically mature seeds.

“Seed maturation” refers to the period starting with fertilization in which metabolizable reserves, e.g., sugars, oligosaccharides, starch, phenolics, amino acids, and proteins, are deposited, with and without vacuole targeting, to various tissues in the seed (grain), e.g., endosperm, testa, aleurone layer, and scutellar epithelium, leading to grain enlargement, grain filling, and ending with grain desiccation.

“Maturation-specific protein promoter” refers to a promoter exhibiting substantially up-regulated activity (greater than 25%) during seed maturation.

“Heterologous nucleic acid” refers to nucleic acid which has been introduced into plant cells from another source, or which is from a plant source, including the same plant source, but which is under the control of a promoter that does not normally regulate expression of the heterologous nucleic acid.

“Heterologous peptide or polypeptide” is a peptide or polypeptide encoded by a heterologous nucleic acid.

As used herein, the terms “native” or “wild-type” relative to a given cell, polypeptide, nucleic acid, trait or phenotype, refers to the form in which that is typically found in nature.

As used herein, the term “purifying” is used interchangeably with the term “isolating” and generally refers to any separation of a particular component from other components of the environment in which it is found or produced. For example, purifying a recombinant protein from plant cells in which it was produced typically means subjecting transgenic protein-containing plant material to separation techniques such as sedimentation, centrifugation, filtration, and chromatography. The results of any such purifying or isolating step(s) may still contain other components as long as the results have less of the other components (“contaminating components”) than before such purifying or isolating step(s).

As used herein, the terms “transformed” or “transgenic” with reference to a host cell means the host cell contains a non-native or heterologous or introduced nucleic acid sequence that is absent from the native host cell. Further, “stably transformed” in the context of the present invention means that the introduced nucleic acid sequence is maintained through two or more generations of the host, which is preferably (but not necessarily) due to integration of the introduced sequence into the host genome.

### Recombinant Human Lactoferrin (LF)

LF is a member of the transferrin family of 80 kDa iron binding proteins, which possess a single polypeptide chain and two iron-binding domains. It is found in high concentration (average 1-2 g/L) in human milk. It is secreted by the salivary and pancreatic glands and in granules of neutrophils. LF is a multifunctional protein with many biological activities, including antimicrobial activities against human pathogens. Other activities of LF include regulation of iron absorption, immune system modulation, and anti-inflammation.

LF has also been shown to act as a growth factor promoting cell growth and delaying apoptosis. In searching for factors in human milk that have cell growth effects, it was found that LF from milk promoted proliferation of rat intestinal cell growth similarly to human colostrum. (Nichols, B L, et al., Human lactoferrin supplementation of infant formulas increases thymidine incorporation into the DNA of rat crypt cells. J Pediatr Gastroenterol Nutr 8: 102-9 (1989); Nichols, B L, et al., Human lactoferrin stimulates thymidine incorporation into DNA of rat crypt cells. Pediatr Res 21: 563-7 (1987).) In addition, when searching for a factor in human milk that can promote bone cell growth, it was found that LF promoted osteoblast growth and delayed apoptosis of bone cells. (Cornish, J., et al., Lactoferrin is a potent regulator of bone cell activity and increases bone formation in vivo. Endocrinology 145: 4366-74 (2004).) Some evidence exists on the stimulatory effects of LF on the proliferation in rat intestinal epithelial cells, mouse 3T3 fibroblasts, rat hepatocytes, human lymphocytes, bovine epithelial cells, human endometrial stroma cells, and mouse embryonic cells. Preliminary studies conducted by the inventors have shown that recombinant human LF induces of proliferation of skin cells, such as keratinocytes, fibroblasts, and skin stem cells.

Two hypotheses have been proposed to explain the cell growth stimulating property of LF. Many of the studies mentioned above showed that holo-LF (iron-bound LF) has a stronger cell growth promoting effect than apo-LF (LF without bound iron). It is believed that through LF's ability to bind and deliver iron, cells are stimulated to proliferate. The second hypothesis is based on the fact that LF binds to receptors on the cell surface, enters the cell cytoplasm, and enters the nuclei to interact with DNA. It is proposed that LF acts in the signal transduction pathway to regulate gene expression and cell development. It is possible that both mechanisms act simultaneously to promote cell growth. The effects of LF on cell growth are different from that of EGF and FGF because LF has a synergistic effect on cell growth with both EGF and FGF.

Using an immunocytochemical approach, LF has been found to be present in the epidermis, sweat grands and follicles. Within the epidermis, LF was found to associate with keratinocytes. (Cumberbatch, M., et al., Regulation of epidermal Langerhans cell migration by lactoferrin. Immunology 100: 21-8 (2000).) A LF cell surface receptor, LRP (LDL receptor-related protein), was found to be involved in regulating smooth muscle cell motility or contractility. (Nassar, T., et al., Binding of urokinase to low density lipoprotein-related receptor (LRP) regulates vascular smooth muscle cell contraction. J Biol Chem 277: 40499-504 (2002).) An in vitro collagen gel assay showed that addition of LF enhanced motile activity of WI-38 human fibroblasts by specifically binding to the cells. (Takayama, Y. and Mizumachi, K., Effects of lactoferrin on collagen gel contractile activity and myosin light chain phosphorylation in human fibroblasts, FESS Lett 508: 111-6 (2001); Takayama, Y., et al., Low density lipoprotein receptor-related protein (LRP) is required for lactoferrin-enhanced collagen gel contractile activity of human fibroblasts. J Biol Chem 278: 22112-8 (2003).) In addition, LF was found to have protective effects against UV-B irradiation-induced corneal epithelial damage in rats (Fujihara, T., et al., Lactoferrin protects against UV-B irradiation-induced corneal epithelial damage in rats. Cornea 19: 207-11 (2000)), implying that LF might play a role in cell repair. Glycosylphosphatidylinositol-anchored LF-binding protein has been shown to be expressed on keratinocytes and fibroblasts in both normal skin and the chronic ulcer. (Damiens, E., et al., Role of heparan sulphate proteoglycans in the regulation of human lactoferrin binding and activity in the MDA-MB-231 breast cancer cell line. Eur J Cell Biol 77: 344-51 (1998).) And heparan sulfate proteoglycans, which have an affinity for LF, have been shown to be a crucial molecule in the formation of human epidermis. (Sher, I., et al., Targeting perlecan in human keratinocytes reveals novel roles for perlecan in epidermal formation. J Biol Chem (2005).) Furthermore, the inventors have discovered that LF significantly increases cell proliferation of human keratinocytes, further indicating the potential functions of LF in wound healing. Finally, because wound healing is commonly impaired by infection and anemia due to iron deficiency or defective iron utilization associated with chronic illnesses (Li, J. and Krisner, R S, Wound Healing Surgery of the Skin, pp. 97-115. Elsevier Mosby (2005)), using LF on wounds is expected to provide an additional benefit in iron delivery and infection control. Therefore, LF is a strong candidate for a wound healing agent because of its activity as a growth factor, iron delivery, anti-infective properties, and synergistic effects with EGF and FGF.

### Benefits of Recombinant LF and LZ Produced in Plants

The use of recombinant human LF and recombinant human LZ produced in plants in accordance with the present invention to manage wound healing, prevent biofilm formation, and promote formation of artificial skin was a result of the consideration of several factors.

First, the LF and LZ must be effective.

Second, the administered rhLF and rhLZ must remain physically stable at the wound site. LF and LZ have been shown to be resistant to protease digestion and stable over a wide range of pHs.

Third, there must be an abundant source of LF and LZ at an affordable cost. For example, when used for research purposes, native hLF costs over $30,000/gram (Sigma Chemicals, St. Louis, Mo.), and its availability is limited. There are abundant source of bovine LF, however, there are concerns regarding its potential allergenicity. Therefore, the use of rhLF and rhLZ are preferred embodiments of the invention.

A particularly preferred option is to produce recombinant human LF (rhLF) in plants, since plant-based production of recombinant proteins is believed to have the capability of producing large quantities at low cost. Previous attempts have been made to produce rhLF in various plants including tobacco, tomato, potato, and corn, but the expression levels were too low. The inventors have successfully expressed rhLF in rice at 0.5% of rice flour weight or over 25% total soluble protein, which is 10- to 100-fold higher than reported results. Therefore, large amounts of rhLF can be produced from transgenic rice. Accordingly, a preferred embodiment of the present invention utilizes rhLF that is expressed in rice grains. Initial biochemical and biophysical studies have shown that rhLF is substantially equivalent to nhLF, and it is anticipated that rhLF will perform as an effective wound healing agent by promoting cell growth with the additional benefit of anti-antimicrobial activity. The present invention therefore focuses on use of rhLF to promote cell growth, and more particularly, to stimulate keratinocyte proliferation, for its potential use in wound healing and in artificial skin production. It is also believed that rhLF will be demonstrated to be an effective anti-microbial agent, and its use in this capacity is also envisioned by the present invention.

Fourth, LF used to manage wound healing must be safe. Recombinant hLF that has the same biochemical and biophysical characteristics as its native counterpart in the human body should provide the safest choice of LF. Since rice is not a known allergen, rhLF produced in rice would not invoke an allergic response as bovine LF or rhLF produced by microorganisms would.

The rhLF produced in rice in accordance with the present invention has the potential to meet all four criteria set forth above; therefore expression of rhLF in rice provides a solid foundation for developing novel compositions and methods for managing wound healing. Furthermore, use of rice as the host for the production of rhLF provides several distinct advantages over the prior art:

Low production cost. Commercial rice grain can be produced at a cost of $0.22/kg. When transgenic rice grain expressing rhLF is produced on a large scale, the cost of commercial rice production and rhLF, before purification, is approximately $0.034/gram.

Mass production possibility. With the current expression of 0.5% flour weight and 10 ton of grain/hectare (ha), over 40 kg of rhLF can be produced from a hectare of rice. Production of 10 tons of rhLF for worldwide demand, requires only 312 ha of rice, which is smaller than a typical rice farm (about 400 ha/farm) in the United States.

Long term grain storage for continuous processing. Rice can be stored at ambient temperatures for more than two years without losing its viability. Rice expressing rhLF has been stored over two years and has not shown any loss or degradation of the recombinant protein.

Rice is a self-pollinating crop which prevents gene flow. A closed rice production system with segregation of the rhLF-rice from any other rice in the area has been effectively utilized by Ventria, and that system was reviewed and approved by the USDA in 1997.

### Production of Recombinant Lactoferrin and Lysozyme

According to one presently preferred embodiment, the present invention utilizes lactoferrin (LF) and lysozyme (LZ) that are recombinantly produced in a host plant seed. Preferably, the LF or LZ expressed comprises about 1% or greater of the total soluble protein in the seed. Thus, for example, the yield of total soluble protein which comprises the LF or LZ targeted for production can be about 3% or greater, about 5% or greater, about 10% or greater, most preferably about 20% or greater, of the total soluble protein found in the recombinantly engineered plant seed.

Preferably, the LF or LZ constitutes at least 0.01 weight percent of the total protein in the harvested seeds. More preferably, the LF or LZ constitutes at least 0.05 weight percent, most preferably at least 0.1 weight percent, of the total protein in the harvested seeds.

An embodiment of the present invention includes a method of producing LF or LZ in plant seeds, comprising the steps of:

(a) transforming a plant cell with a chimeric gene comprising
(i) a maturation-specific protein promoter from a plant,
(ii) a first nucleic acid sequence, operably linked to the promoter, encoding a seed-specific signal sequence capable of targeting a polypeptide linked thereto to seed endosperm, and
(iii) a second nucleic acid sequence, linked in translation frame with the first nucleic acid sequence, encoding LF or LZ, wherein the first nucleic acid sequence and the second nucleic acid sequence together encode a fusion protein comprising an N-terminal signal sequence and the LF or LZ;

(b) growing a plant from the transformed plant cell for a time sufficient to produce seeds containing the LF or LZ; and

(c) harvesting the seeds from the plant.

Preferably, the plant is a monocot plant. More preferably, the plant is a cereal, preferably selected from the group consisting of rice, barley, wheat, oat, rye, corn, millet, triticale and sorghum.

The promoter is preferably from a maturation-specific monocot plant storage protein or an aleurone- or embryo-specific monocot plant gene. Other promoters may be used, however, and the choice of a suitable promoter is within the skill of those in the art. More preferably, the promoter is a member selected from the group consisting of rice globulins, glutelins, oryzins and prolamines, barley hordeins, wheat gliadins and glutenins, maize zeins and glutelins, oat glutelins, sorghum kafirins, millet pennisetins, rye secalins, lipid transfer protein Ltp1, chitinase Chi26 and Em protein Emp1. Most preferably, the promoter is selected from the group consisting of rice globulin Glb promoter and rice glutelin Gt1 promoter.

The seed-specific signal sequence is preferably from a monocot plant, although other signal sequences that target polypeptides to seed endosperm may be utilized. Preferably, the monocot plant seed-specific signal sequence is associated with a gene selected from the group consisting of glutelins, prolamines, hordeins, gliadins, glutenins, zeins, albumin, globulin, ADP glucose pyrophosphorylase, starch synthase, branching enzyme, Em, and lea. More preferably, the monocot plant seed-specific signal sequence is associated with a gene selected from the group consisting of α-amylase, protease, carboxypeptidase, endoprotease, ribonuclease, DNase/RNase, (1-3)-β-glucanase, (1-3)(1-4)-β-glucanase, esterase, acid phosphatase, pentosamine, endoxylanase, β-xylopyranosidase, arabinofuranosidase, β-glucosidase, (1-6)-β-glucanase, perioxidase, and lysophospholipase. Most preferably, the monocot plant seed-specific signal sequence is a rice glutelin Gt1 signal sequence.

As will be understood by those of skill in the art, in some cases it may be advantageous to use nucleotide sequences possessing non-naturally occurring codons. Codons preferred by a particular eukaryotic host can be selected, for example, to increase the rate of expression or to produce recombinant RNA transcripts having desirable properties, such as a longer half-life, than transcripts produced from naturally occurring sequence. As an example, it has been shown that codons for genes expressed in rice are rich in guanine (G) or cytosine (C) in the third codon position (Huang et al., J. CAASS 1: 73-86, 1990). Changing low G+C content to a high G+C content has been found to increase the expression levels of foreign protein genes in barley grains (Horvath et al., Proc. Natl. Acad. Sci. USA 97: 1914-19, 2000). If a rice plant is selected, the genes employed in the present invention may be based on the rice gene codon bias (Huang et al., supra) along with the appropriate restriction sites for gene cloning. These codon-optimized genes may be linked to regulatory and secretion sequences for seed-directed expression and these chimeric genes then inserted into the appropriate plant transformation vectors.

The method used for transformation of host plant cells is not critical to the present invention. For commercialization of the heterologous peptide or polypeptide expressed in accordance with the invention, the transformation of the plant is preferably permanent, i.e. by integration of the introduced expression constructs into the host plant genome, so that the introduced constructs are passed onto successive plant generations. The skilled artisan will recognize that a wide variety of transformation techniques exist in the art, and new techniques are continually becoming available.

Any technique that is suitable for the target host plant may be employed within the scope of the present invention. For example, the constructs can be introduced in a variety of forms including, but not limited to, as a strand of DNA, in a plasmid, or in an artificial chromosome. The introduction of the constructs into the target plant cells can be accomplished by a variety of techniques, including, but not limited to calcium-phosphate-DNA co-precipitation, electroporation, microinjection, Agrobacterium-mediated transformation, liposome-mediated transformation, protoplast fusion or microprojectile bombardment. The skilled artisan can refer to the literature for details and select suitable techniques for use in the methods of the present invention.

Transformed plant cells are screened for the ability to be cultured in selective media having a threshold concentration of a selective agent. Plant cells that grow on or in the selective media are typically transferred to a fresh supply of the same media and cultured again. The explants are then cultured under regeneration conditions to produce regenerated plant shoots. After shoots form, the shoots can be transferred to a selective rooting medium to provide a complete plantlet. The plantlet may then be grown to provide seed, cuttings, or the like for propagating the transformed plants.

The expression of the heterologous peptide or polypeptide may be confirmed using standard analytical techniques such as Western blot, ELISA, PCR, HPLC, NMR, or mass spectroscopy, together with assays for a biological activity specific to the particular protein being expressed.

### Compositions Containing Lactoferrin, and Methods of Making and Using Same

The present invention provides compositions for use in wound treatment that contain lactoferrin (LF) in an amount effective for promoting cell growth and migration, and preferably directly promoting proliferation of skin cells, where the skin cells may include, without limitation, keratinocytes, fibroblasts, and skin stem cells. Preferably, the LF is provided in an amount of from about 0.01% to about 20%, and more preferably in an amount of from about 0.1% to about 10%. According to a preferred embodiment, the compositions also possess antimicrobial activity, which may also be attributed to the LF.

According to a particularly preferred embodiment of the present invention, the composition also includes LZ, which provides additional antimicrobial activity. Preferably, the LZ is provided in an amount of from about 0.001% to about 20%, and more preferably in an amount of from about 0.01% to about 5%.

Preferably, the LF is provided in the form of rhLF, and most preferably, the rhLF is produced in a plant, preferably a monocot plant, such as rice. Preferably, the LZ is also provided in the form of rhLZ, and most preferably, the rhLZ is also produced in a plant, preferably a monocot plant, such as rice. However, use of other, alternative forms of LF and LZ are also envisioned in accordance with the present invention, and are considered to be within the scope of the compositions and topical formulations of the present invention. Such LF and LZ may be native or recombinant, and may be derived from any plant or animal source, including bovine LF and chicken egg LZ, bovine rhLF and rhLZ, and native hLF and hLZ.

In one embodiment, the compositions of the invention containing LF, or a combination of LF and LZ, are delivered to the affected area of the skin in a pharmaceutically acceptable topical carrier. As used herein, a pharmaceutically acceptable topical carrier is any pharmaceutically acceptable formulation that can be applied to the skin surface for topical, dermal, intradermal, or transdermal delivery of the active agent(s). The formulations of this invention may be provided in any convenient semisolid or fluid form, such as pastes, creams, gels, aerosols, solutions or dispersions.

The topical formulations of the invention can further comprise pharmaceutically acceptable excipients, including, but not limited to, protectives and adsorbents (such as dusting powders, zinc sterate, collodion, dimethicone, silicones, zinc carbonate, aloe vera gel and other aloe products, vitamin E oil, allatoin, glycerin, petrolatum, and zinc oxide); demulcents (such as benzoin, hydroxypropyl cellulose, hydroxypropyl methylcellulose, and polyvinyl alcohol); emollients (such as animal and vegetable fats and oils, myristyl alcohol, alum, and aluminum acetate); preservatives (such as quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenylethyl alcohol, and benzyl alcohol; antibacterial esters, for example, esters of parahydroxybenzoic acid; and other anti-microbial agents such as chlorhexidine, chlorocresol, benzoic acid and polymyxin); stabilizers; antioxidants (such as ascorbic acid and its esters, sodium bisulfite, butylated hydroxytoluene, butylated hydroxyanisole, tocopherols, and chelating agents like EDTA and citric acid); moisturizers (such as glycerin, sorbitol, polyethylene glycols, urea, and propylene glycol); buffering agents (such as acetate buffers, citrate buffers, phosphate buffers, lactic acid buffers, and borate buffers); solubilizing agents (such as quaternary ammonium chlorides, cyclodextrins, benzyl benzoate, lecithin, and polysorbates); skin-penetration agents (such as ethyl alcohol, isopropyl alcohol, octylphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone); and surfactants.

Other beneficial pharmaceutical additives may be optionally provided in conjunction with the topical formulations of the present invention, including any compounds known or believed to be useful in the treatment of acne, aging, age spots, sunburns, inflammation, rosacea, psoriasis, eczematous dermatitis (such as allergic contact dermatitis, atopic dermatitis, nummular eczematous dermatitis, seborrheic dermatitis, vesicular palmoplantar eczema), bites, stings, and infestations. The optional additives may also included compounds known or believed to be useful in the treatment of skin lesions, disorders, and infections caused by Gram-negative and Gram-positive bacteria, mycobacteria, fungi, yeast, and/or viruses, which may be attributable to, for example, sexually transmitted diseases. Further, these optional pharmaceutical additives may also include antibiotics, pain relievers, and other compounds known to those skilled in the art to be useful in wound and burn treatment.

Topical formulations of the invention are prepared by mixing the compositions containing LF, or a combination of LF and LZ, with the topical carrier according to well-known methods in the art. The topical carriers useful for delivery of compositions of the invention can be any carrier known in the art for topically administering pharmaceuticals, including, but not limited to, pharmaceutically acceptable solvents, such as a polyalcohol or water; emulsions (either oil-in-water or water-in-oil emulsions), such as creams or lotions; micro emulsions; gels; ointments; liposomes; powders; and aqueous solutions or suspensions.

The compositions and topical formulations of the present invention are useful for treating skin disorders, conditions, and inflammation according to the methods of treatment of the present invention. Preferably, the compositions are applied to the skin via application of a safe and effective amount of the topical formulation to treat the skin. Application may be accomplished by any suitable means such as by manual spreading or rubbing, applicator pads, brushes, aerosol spray, pump spray, or any other suitable means which assures that the affected skin surface will be entirely covered. For example, it can be directly applied to the skin being treated, or used to coat a dressing that is then be placed on the skin. The dose range, rate and duration of treatment will vary with and depend upon the type and severity of the skin condition/disorder, the area of the body which is afflicted, patient response and like factors, as will be understood by those of skill in the art.

The compositions and topical formulations of the present invention are also useful for treating wounds according to the methods of treatment of the present invention. Preferably, the compositions are applied to a wound via application of a safe and effective amount of the topical formulation to treat the wound. Application may be accomplished by any suitable means such as by manual spreading or rubbing, applicator pads, brushes, aerosol spray, pump spray, or any other suitable means which assures that the wound surface will be entirely covered. For example, it can be directly applied to the wound site or used to coat fibers of an absorbent dressing to form a wound healing bandage which may then be placed on a wound. The dose range, rate and duration of treatment will vary with and depend upon the type and severity of the wound, the area of the body which is afflicted, patient response and like factors, as will be understood by those of skill in the art.

### Artificial Skin Formation and Methods of Treating Burns Using Same

The present invention also relates to the use of lactoferrin (LF) in producing artificial skin, because of its ability to increase cell growth/migration and its capacity for antimicrobial activity. The compositions of the present invention containing LF, or a combination of LF and LZ, are useful in producing artificial skin for use in treating burns, and for application to burn wounds, optionally in conjunction with artificial skin, in order to promote healing.

The present invention provides compositions for use in burn treatment that contain lactoferrin (LF) in an amount effective for promoting cell growth and migration, particularly directly promoting proliferation of keratinocytes. The compositions may be provided in vitro to promote the growth of artificial skin, or in vivo to treat burn wounds. Preferably, the LF is provided in an amount of from about 0.01% to about 20%, and more preferably in an amount of from about 0.1% to about 10%. According to a preferred embodiment, the compositions also possess antimicrobial activity, which may also be attributed to the LF.

According to a particularly preferred embodiment of the present invention, the composition also includes LZ, which provides additional antimicrobial activity. Preferably, the LZ is provided in an amount of from about 0.001% to about 20%, and more preferably in an amount of from about 0.01% to about 5%.

Preferably, the LF is provided in the form of rhLF, and most preferably, the rhLF is produced in a plant, preferably a monocot plant, such as rice. Preferably, the LZ is also provided in the form of rhLZ, and most preferably, the rhLZ is also produced in a plant, preferably a monocot plant, such as rice. However, use of other, alternative forms of LF and LZ are also envisioned in accordance with the present invention, and are considered to be within the scope of the compositions and topical formulations of the present invention. Such LF and LZ may be native or recombinant, and may be derived from any plant or animal source, including bovine LF and chicken egg LZ, bovine rhLF and rhLZ, and native hLF and hLZ.

In one embodiment, the compositions of the invention containing LF, or a combination of LF and LZ, are used to aid in the production of artificial skin. The term artificial skin, as used herein, may refer to artificial dermis (formed using fibroblasts), artificial epidermis (formed using keratinocytes), or an artificial skin comprising both an artificial dermal and an artificial epidermal layer.

Artificial skin is generally produced by constructing a biodegradable scaffolding on which skin cells can be grown. Fibroblasts seeded onto an appropriate scaffolding will proliferate and arrange themselves to form an artificial dermal layer. Keratinocytes seeded onto an appropriate scaffolding will proliferate and arrange themselves to form an artificial epidermal layer. Alternatively, after the artificial dermal layer has been allowed several weeks to form, keratinocytes may be seeded directly on to the artificial dermal tissue, to create an artificial skin including both a dermal and an epidermal layer.

The compositions of the present invention, which may include LF, or a combination of LF and LZ, may be beneficially incorporated into the production of the artificial skin, both for the production of the dermal layer and the epidermal layer, in order to promote growth and migration of the fibroblasts and keratinocytes. The compositions and methods of the present invention thereby improve the process for producing artificial skin. The resulting artificial skin may be used to treat burns and other wounds.

In another embodiment, the compositions and topical formulations of the present invention are used to promote healing of burns, optionally in conjunction with the application of artificial skin.

### Alternative Embodiments

Additional uses for the compositions and methods of the present invention, beyond treatment of skin disorders/conditions, prevention of biofilm formation, treatment of acute and chronic wounds, and treatment of burns and formation of artificial skin, are also envisioned.

It will, of course, be appreciated that the above description has been given by way of example only and that modifications in detail may be made within the scope of the present invention.

### EXAMPLES

The present invention will now be described with reference to the following examples.

1. Production of rhLF in Rice

1.1. Construction of Expression Vector of a Synthetic Human Lactoferrin Gene

The human lactoferrin (hLF) gene was synthesized based on the codon-preference of rice genes (called codon-optimization). By codon-optimization either an A or a T at the third position of the codons was changed to G or C. Of the 692 codons for the mature peptide of the hLF gene, 413 codons were changed but the amino acid sequence of the synthetic hLF gene remained the same. The codon-optimized hLF gene was linked to a rice endosperm-specific glutelin (Gt1) promoter and the NOS terminator. The glutelin signal peptide was used with the expectation that recombinant human lactoferrin (rhLF) would be targeted to the protein bodies within the endosperm cells thus preventing the degradation of rhLF in rice cells.

1.2. High Level Expression of rhLF in Transgenic Rice Grains

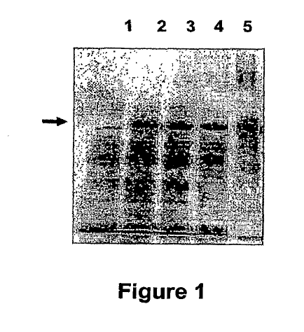

Transgenic plants were produced (n=108) and transgenic grains were harvested from 56 fertile plants. Out of these, grain from 18 plants expressed moderate to high levels of rhLF. Western blot analysis showed that the rhLF had a molecular weight similar to that of native hLF. Total soluble proteins extracted from the rice grains were also analyzed by SOS-PAGE (FIG. 1). Among the protein bands, the rhLF band was the strongest in line LF164, indicating that rhLF is the most abundant soluble protein. Quantitative analysis indicated that up to 25% of soluble protein in line LF164 was rhLF. The expression levels among the independent lines varied from 10 to 100 μg/grain (i.e., 0.5 to 5 g/kg dehusked rice).

The best performing line (LF164) was selected for further analysis. Homozygous lines were selected based on rhLF expression analysis and were advanced. Southern blot analysis was used to examine the stability of transgenes over the generations. All bands were inherited from R₀ to R₄ generations as a single linkage unit or single locus and stable inheritance of the hLF gene was observed through consecutive generations. ELISA was used to examine the stability of rhLF expression over generations. The expression level remains stable at 5.0 g rhLF/kg of dehusked rice grains.

1.3. Purification of rhLF from Transgenic Rice Grains and its Biological Properties

Recombinant hLF from transgenic rice grains was purified to homogeneity using an SP-Sepharose column when conducted in small scale. The N-terminal sequence of rhLF was identical to the corresponding region of hLF, indicating that the rice signal peptidase recognized and cleaved at the junction between the Gt1 signal peptide sequence and the mature rhLF peptide. The isoelectric point (pl) of hLF and rhLF was similar indicating that both have similar surface charges.

The recombinant hLF derived from transgenic rice grains was found to be glycosylated by glycan analysis. Analysis shows that the purified rhLF contains xylose but lacks sialic acid, which is consistent with plant post-translational modification patterns.

1.4. pH Dependent Iron Uptake and Release of rhLF, and Iron Saturation of rhLF

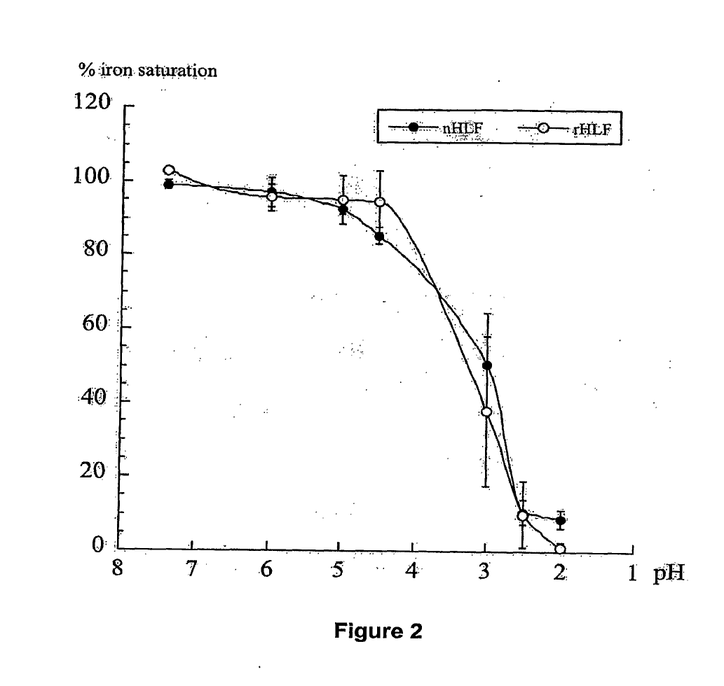

Both native human lactoferrin (nhLF) and rhLF can reach iron-saturation by picking up iron from the solution to form holo-LF. The stability of iron-binding by rhLF toward a low pH was analyzed and compared to that of nhLF (FIG. 2). Iron release began around pH 4 and was completed around pH 2 this was similar for both proteins.

The iron saturation level of rhLF purified from ion exchange column is approximately 50%. To prepare holo-rhLF (>90% iron saturation), rhLF purified from above step was mixed with a iron solution at molar ratio of 1:6 (1 LF:6 iron). The mixture was left overnight at 4° C. to allow rhLF to bind iron. Excess iron was removed by using membranes via ultrafiltration. The iron-saturated rhLF was concentrated and the NaCl concentration reduced via diafiltration with 50 mM sodium chloride. This was performed using Millipore S10 membranes of 30,000 Nominal Molecular Weight Cutoff Membranes. The protein solution was centrifuged to remove any precipitate that formed and was then filtered via 0.45 micron and 0.2 micron filters prior to drying.

1.5. Production of Rice Expressing Recombinant Human Lactoferrin

Over 250 kg of rice (LF164) were produced in 2005 for use in the compositions and methods of the present invention. However, it is important to produce more rice, because it takes 6 months to produce one crop. Field planting will be carried out using procedures established by Ventria Bioscience and approved by USDA. LF164 will be planted on one (1) acre of rice land, and it is expected that a harvest of about 2000 kg of rice will be generated. Since rice planting is standard to those skilled in the art, the details regarding the rice planting are omitted here.

1.6. Process to Generate Recombinant Human Holo-Lactoferrin (Holo-rhLF)

Paddy rice expressing rhLF will be dehulled using a dehuller (Rice Mill, PS-160, Rimac, Fla.). The rice will be ground to flour (average of 100 mesh) using a hammer mill (8WA, Schutte-Buffalo, N.Y.). The flour will be used as starting material throughout the process and preparation of rhLF. Rice flour will be mixed in a specific buffer: flour ratio of 1 to 10 (1 kg flour to 10 L extraction buffer). For pilot scale work, 2 Kg of flour will be mixed with 20 L of extraction buffer (0.3 M NaCl/0.02 M NaPO₄, pH 6.5). The flour will be mixed for a minimum of 60 minutes and then settled. The supernatant will then be clarified using a filter press (Ertel Alsop, Kingston, N.Y.).

1.7. Recombinant hLF Purification Via Cation-Exchange Chromatography

It has been shown that ion-exchange resin, SPFF, is an effective resin for binding rhLF. A column will be packed with SP-Sepharose big bead media (Amersham Pharmacia Biotech, NJ) and evaluated with standard Height Equivalent to a Theoretical Plate (HETP) using 1.0% acetone. An average linear flow of 200-400 cm/hour will be used during purification. Packing and cleaning will be performed as per the manufacturer's specification. The filtrate/concentrate will be loaded onto the SP-Sepharose media where the rhLF will be bound to the media. The column will be washed with water containing 0.3 M NaCl until the absorbance at 280 nm returns to baseline, as measured by an in-line UV monitor. Recombinant hLF will be eluted with 0.8 M NaCl solution as a step elution; the entire peak, as measured by UV absorbance, will be collected as the purified rhLF.

1.8. Lyophilization

The liquid product will be loaded on a Virtus 35 freeze dryer and frozen to a minimum product temperature of −20° C. The drying process will take place with the shelf temperature at 5° C. and then the final drying will take place for a minimum of 2 hours with the product at 25° C.

1.9. Quantification and Characterization of rhLF

The quantity and purity of the rhLF will be determined by SDS-PAGE analysis, ELISA and using a BioCAD 60 (PerSeptive Biosystems, MA). Reverse phase separation will be carried out using a Zorbax SB-C8 4.6×150 mm (5 μm particles, 80 Å pores) analytical column (Agilent, Calif.) at a flow rate of 1 ml/min with detection at 210 nm. Separation will be accomplished with linear AB gradients where eluent A will be aqueous trifluoroacetic acid (0.2%) and eluent B will be acetonitrile with 0.2% trifluoroacetic acid. About 25 μg rhLF will be injected for each analysis.

2. Effects of rhLF

Keratinocytes and fibroblasts are the two major types of cells in the skin. These examples measure the effects of using in vitro cell cultures supplemented with rhLF at various concentrations. Since the rhLF is a member of the transferrin family and various studies have shown growth factor-like effects on cell proliferation in certain cell lines from other systems, the first step was to explore the potential mechanisms involved, and to test the effects of rhLF in the presence or absence of transferrin and growth factors important in wound healing.

2.1. Antimicrobial Effect of rhLF

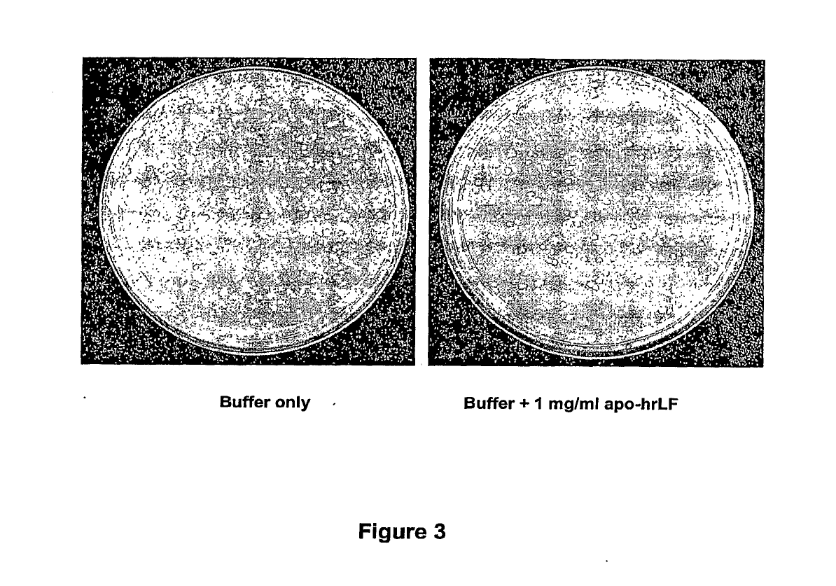

The antimicrobial activity of recombinant human lactoferrin was measured using E. coli as substrate. Culture cells of E. coli K12 were prepared from cultured plates. About 10⁵ CFU of E. coli in one mL was mixed with 1 mg of rhLF; the control contained no lactoferrin. The mixture was incubated at 37° C. for 120 minutes with shaking at 250 rpm. Five μL of mixture was then plated and colony forming units were determined. As can be seen in FIG. 3, there is marked reduction in colony forming units in the culture with added rhLF.

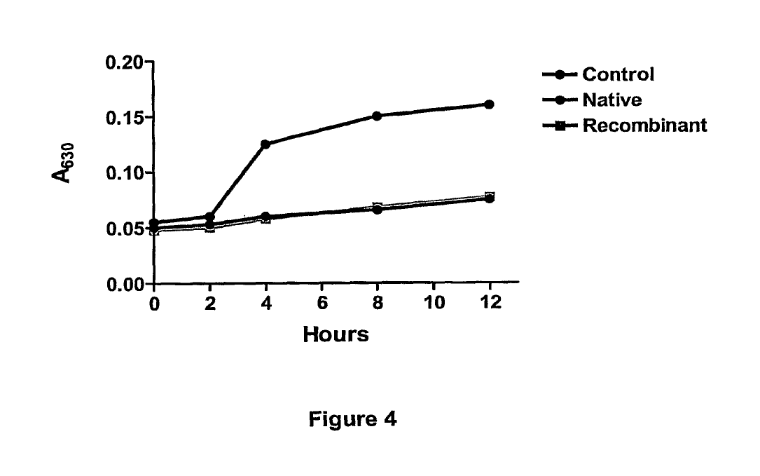

To compare the effect of rhLF and native hLF on E. coli growth, another experiment was carried out with three groups: negative control (growth media only), positive control (growth media with native hLF) and treatment (growth media with rhLF), and the results are shown in FIG. 4. There was a marked increase of E. coli growth in growth media while E. coli growth was much slower in treatments with native and recombinant human lactoferrin indicating that recombinant human lactoferrin has the same effect as the native lactoferrin in controlling E. coli growth in culture media.

2.2. Effect of rhLF on HT29 Proliferation

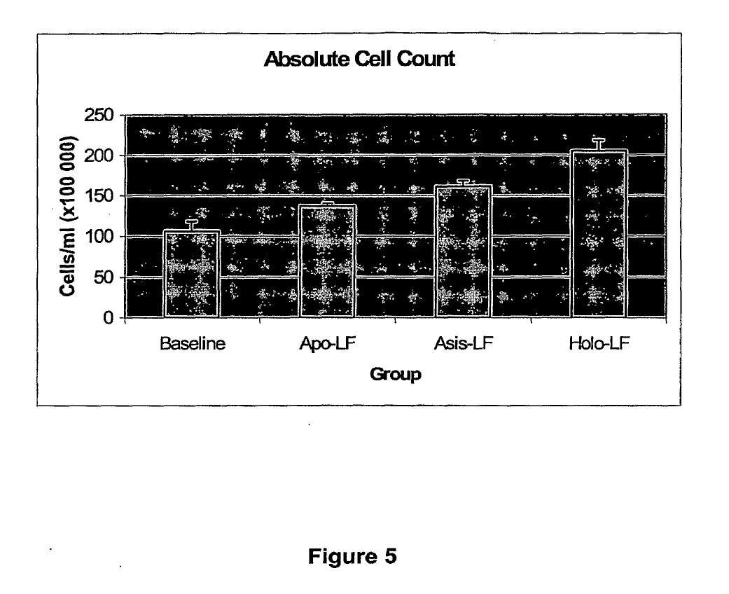

HT29 cells were seeded and grown in MEM containing 5% fetal calf serum alone or MEM containing 5% fetal calf serum containing 1 mg/mL of apo-, asis-, or holo-LF. Cell viability, determined by the ability to exclude 0.2% trypan blue, was greater than 90%. Cells were counted using a hemocytometer. Cells present in the supernatant were included in the total cell count by centrifugation of the supernatant. As seen in FIG. 5, HT29 cell density in media with holo-LF is approximately twice that of the control. This indicates that holo-rhLF has a strong cell growth promoting effect.

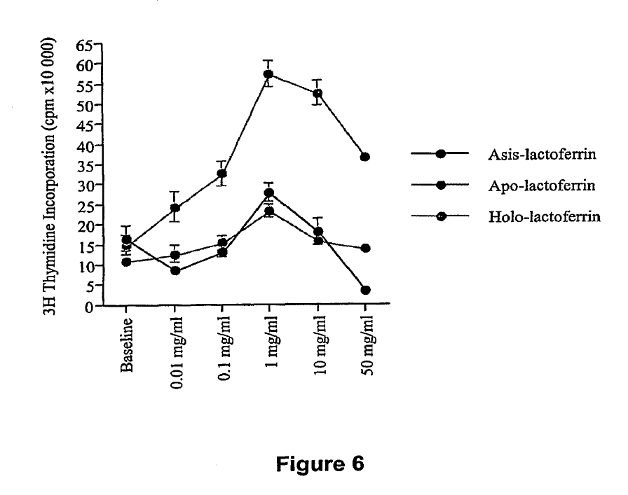

Proliferation studies of HT29 cells were performed in subconfluent cell populations. To assess the percentage of cells entering DNA synthesis, [³H] thymidine incorporation was assessed by pulsing the cells with [³H] thymidine (2 μCi/well) 24 hours after the addition of the test factors and cells were left for a further 24 hours. For each condition, the stimulatory or inhibitory effect of the solutions was measured in duplicate in six separate wells. Cell viability, determined by the ability to exclude 0.2% trypan blue, was greater than 90%. FIG. 6 shows the proliferative response in HT-29 cells when exposed to the three forms of lactoferrin (asis-, apo-, and holo-). The figure also reveals a “bell shaped” dose response curve. Of the three forms tested, the holo-rhLF showed the greatest proliferative activity.

2.3. The Effect of rhLF on Human Keratinocyte Proliferation

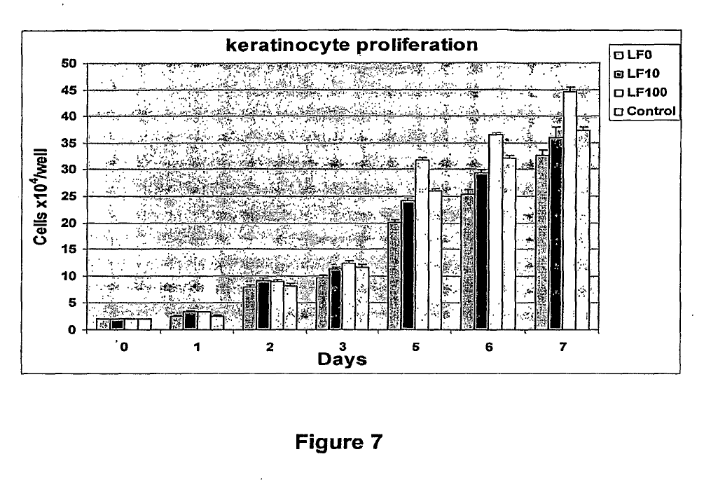

Various concentrations of holo-rhLF (0-100 μg/mL; denoted LF₀, LF₁₀, LF₁₀₀) were used to test its growth promoting effects on normal human skin keratinocytes. (See also Chen et al., “Lactoferrin promotes human skin keratinocyte proliferation,” presented on May 2, 2006 at 19^{th} Annual Symposium on Advanced Wound Care.) The keratinocytes were plated in 12-well plates at day 0 and cells were grown in EpiLife base medium with human keratinocyte growth supplements (HKGS; Cascade) without transferrin, but with varying concentrations of holo-rhLF for 7 days. The control was the media with complete HKGS (with transferrin). Cells were counted by trypsan blue exclusion assay in triplicate. One way ANOVA (analysis of variant) was used for statistical analysis and a P value <0.05 was considered significant. FIG. 7 shows the significant effects of LF on skin keratinocyte proliferation at 10 μg/ml of LF on days 5-6 as compared to the cells grown in the medium without LF (LFₒ; P<0.01). And even stronger stimulatory effects of LF on keratinocytes were observed at the concentration of 100 μg/ml on days 5-7 (P<0.001) as compared to the other test groups.

2.4. The Effects of rhLF on HT29 Cell Migration

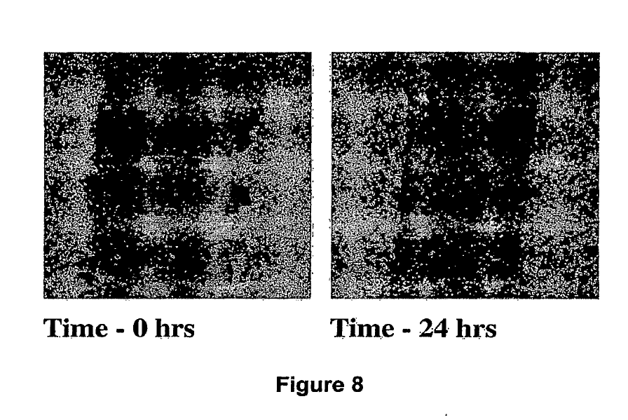

One of the earliest repair responses following injury is migration of surviving cells over the wounded area to re-establish epithelial integrity. HT29, were grown to confluence in 6-well plates in MEM containing 10% fetal calf serum. The mono-layers were then wounded by scraping a disposable pipette tip across the dishes, washed with fresh serum-free medium and cultured in serum-free medium in the presence of various doses of lactoferrin. The rate of movement of the anterior edges of the wounded monolayers was determined by taking serial photomicrographs at various times after wounding. An inverted microscope (Nikon TS100) and a NIKON Coolpix 800 digital camera with 100-fold magnification were used to obtain photomicrographs. Identical regions were examined at each time point by pre-marking the base of the plates to facilitate alignment. Twenty measurements per field were performed by placing a transparent grid over the photograph and measuring the distance moved from the original wound line. Each wound was examined in at least three different regions and are expressed as mean+/−SEM of three separate experiments. All forms of rhLF (asis-, apo-, and holo-) showed a similar dose dependant increase in cell migration (FIG. 8). Maximal stimulation was seen at 1.0 μg/ml holo-rhLF. Note that these concentrations are much lower (μg/ml) than concentrations used for proliferation assay (mg/ml).

### 3. Antimicrobial Effect of Recombinant Human Lysozyme

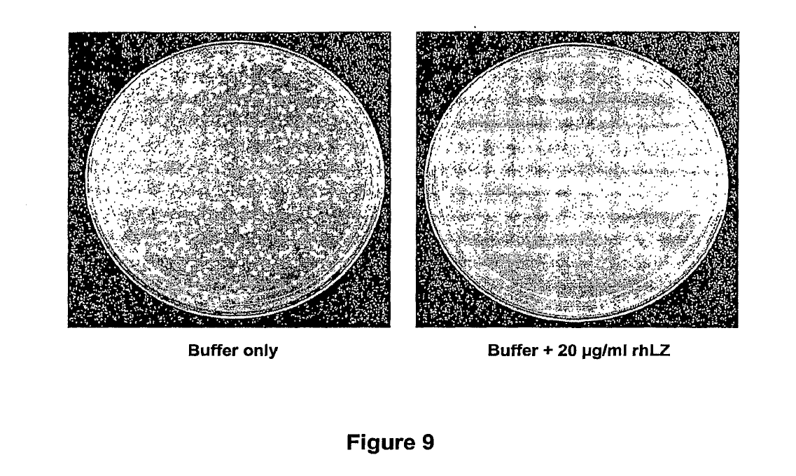

The antimicrobial activity of recombinant human lysozyme was measured using E. coli as substrate. Culture cells of E. coli K12 were prepared from cultured plates. About 10⁵ CFU of E. coli in one mL was mixed with 20 μg of rhLZ; the control contained no lysozyme. The mixture was incubated at 37° C. for 120 minutes with shaking at 250 rpm. Five μL of mixture was then plated and colony forming units were determined. As can be seen from FIG. 9, there is marked reduction in colony forming units in the culture with added rhLZ.

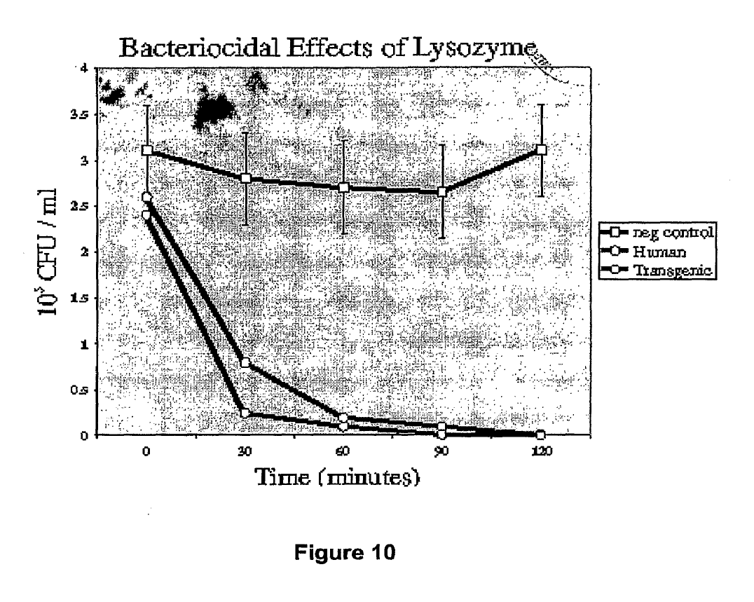

To compare the antibacterial effect of rhLZ and native hLZ, the same experiment was repeated with three groups: negative control (buffer only), positive control (buffer with native hLZ) and treatment (buffer with rhLZ), as shown in FIG. 10. There was no reduction in colony forming units in negative control while there was a significant decrease of E. coli in the positive control. At 60 min incubation time, colony forming units were reduced to 100 and approached zero at 120 min. Treatment with rhLZ had the same effect as the positive control, indicating that both rhLZ and hLZ have the bactericidal activity.

### 4. Protocol to Test Effect of Recombinant Human Lactoferrin on Rosacea

To determine the effectiveness of rhLF in the treatment of rosacea, a 12 week double-blind study will be conducted during which the compositions and/or formulations of the present invention will be applied to the affected skin area twice daily by a group of 40 patients suffering from rosacea. The patients will be eligible for participation in the study if they are between the ages of 18 and 70, and suffering from mild to moderate inflammatory rosacea. The patients will be divided into a two groups evenly matched in terms of age and clinical severity. Twenty patients will apply rhLF in a topical vehicle, and the other 20 patients will apply only the topical vehicle (placebo).

Clinical evaluations of the affected areas will be performed on each patient at the start of the study, and at weeks 3, 6, and 12. The efficacy of the rhLF in the treatment of rosacea will be assessed relative to the improvement from baseline. Factors such as redness, number of lesions, sensory perceptions, desquammation (peeling), and overall assessment by a physician will be considered, as will responses to questionnaires completed by the patients.

### 5. Protocol to Test Effect of Recombinant Human Lactoferrin on Acne

To determine the effectiveness of rhLF in the treatment of acne, a 12 week double-blind study will be conducted during which the compositions and/or formulations of the present invention will be applied to the affected skin area twice daily by a group of 40 patients suffering from acne. The patients will be eligible for participation in the study if they are between the ages of 18 and 70, and suffering from mild to moderate acne with surface pustules and papules (no female hormonal acne). The patients will be divided into a two groups evenly matched in terms of age and clinical severity. Twenty patients will apply rhLF in a topical vehicle, and the other 20 patients will apply only the topical vehicle (placebo).

Clinical evaluations of the affected areas will be performed on each patient at the start of the study, and at weeks 3, 6, and 12. The efficacy of the rhLF in the treatment of acne will be assessed relative to the improvement from baseline. Factors such as redness, number of lesions, sensory perceptions, desquammation (peeling), and overall assessment by a physician will be considered, as will responses to questionnaires completed by the patients.

Throughout this application, various publications have been cited. The disclosures of these publications in their entireties are hereby incorporated by reference into this application, in order to more fully describe the state of the art to which this invention pertains.

While the present invention has been described for what are presently considered the preferred embodiments, the invention is not so limited. To the contrary, the invention is intended to cover various modifications and equivalent arrangements included within the spirit and scope of the detailed description provided above.

1. A topical formulation comprising lactoferrin in an amount effective for directly promoting cell growth and migration.

2. The topical formulation of claim 1, wherein the lactoferrin is isolated from milk or recombinantly-produced.

3. The topical formulation of claim 2, wherein the lactoferrin is isolated from milk derived from one or more human, bovine, porcine, and goat sources.

4. The topical formulation of claim 2, wherein the lactoferrin is recombinantly-produced.

5. The topical formulation of claim 4, wherein the lactoferrin is recombinantly-produced in plant cells.

6. The topical formulation of claim 5, wherein the recombinantly-produced lactoferrin is obtained from monocot seeds.

7. The topical formulation of claim 4, where the recombinantly-produced lactoferrin is a human lactoferrin.

8. The topical formulation of claim 1, wherein the lactoferrin is provided in an amount of from about 0.01% to about 20% by weight.

9. The topical formulation of claim 8, wherein the lactoferrin is provided in an amount of from about 0.1% to about 10% by weight.

10. The topical formulation of claim 1, wherein the composition further comprises lysozyme in an antimicrobially effective amount.

11. The topical formulation of claim 10, wherein the lysozyme is isolated from milk or eggs, or is recombinantly-produced.

12. The topical formulation of claim 11, wherein the lysozyme is isolated from milk derived from one or more human, bovine, porcine, and goat sources.

13. The topical formulation of claim 11, wherein the lysozyme is isolated from chicken egg whites.

14. The topical formulation of claim 11, wherein the lysozyme is recombinantly-produced.

15. The topical formulation of claim 14, wherein the lysozyme is recombinantly-produced in plant cells.

16. The topical formulation of claim 15, wherein the recombinantly-produced lysozyme is obtained from monocot seeds.

17. The topical formulation of claim 4, where the recombinantly-produced lysozyme is a human lysozyme.

18. The topical formulation of claim 10, wherein the lysozyme is provided in an amount of from about 0.001% to about 20% by weight.

19. The topical formulation of claim 18, wherein the lysozyme is provided in an amount of from about 0.01% to about 5% by weight.

20. The topical formulation of claim 1, further comprising one or more additional compounds useful for treating conditions selected from the group consisting of acne, aging, age spots, sunburns, inflammation, rosacea, psoriasis, eczematous dermatitis, allergic contact dermatitis, atopic dermatitis, nummular eczematous dermatitis, seborrheic dermatitis, vesicular palmoplantar eczema, bites, stings, and infestations.

21. The topical formulation of claim 1, further comprising one or more additional compounds useful for treating conditions selected from the group consisting of lesions, disorders, and infections caused by Gram-negative and Gram-positive bacteria, mycobacteria, fungi, yeast, and viruses.

22. The topical formulation of claim 1, further comprising one or more compounds useful for wound and burn treatment.

23. The topical formulation of claim 22, wherein the compounds useful for wound and burn treatment are selected from the group consisting of antibiotics and pain relievers.

24. The topical formulation of claim 1, wherein the topical formulation is provided as a pharmaceutically-acceptable formulation selected from the group consisting of dermal, intradermal, and transdermal formulations.

25. The topical formulation of claim 24, wherein the pharmaceutically-acceptable formulation is provided in a form selected from the group consisting of a semisolid, a fluid, a paste, a cream, a gel, an aerosol, a solution, and a dispersion.

26. The topical formulation of claim 1, wherein the topical formulation directly promotes skin cell proliferation.

27. The topical formulation of claim 26, wherein the skin cells being proliferated are selected from the group consisting of keratinocytes, fibroblasts, and skin stem cells.

28. A method of promoting skin cell proliferation, including the step of contacting skin cells with an amount of lactoferrin effective to directly promote skin cell proliferation.

29. The method of claim 28, further comprising the step of contacting said skin cells with an antimicrobially-effective amount of lysozyme.

30. The method of claim 29, wherein the skin cells are selected from the group consisting of keratinocytes, fibroblasts, and skin stem cells.

31. A method of treating a skin condition selected from the group consisting of acne, aging, age spots, sunburns, inflammation, rosacea, psoriasis, eczematous dermatitis, allergic contact dermatitis, atopic dermatitis, nummular eczematous dermatitis, seborrheic dermatitis, vesicular palmoplantar eczema, bites, stings, and infestations, comprising the step of applying the topical formulation of claim 1.

32. A method of treating skin lesions and/or infections, comprising the step of applying the topical formulation of claim 1.

33. The method of claim 32, wherein the skin disorder is caused by an underlying condition selected from the group consisting of sexually transmitted diseases, wounds, and burns.

34. The method of claim 33, wherein the wounds are acute or chronic surgical wounds.

35. The method of claim 33, wherein the underlying condition is associated with one or more agents selected from the group consisting of Gram-negative bacteria, Gram-positive bacteria, mycobacteria, fungi, yeast, and viruses.

36. A method of promoting wound healing, comprising the step of applying the topical formulation of claim 1.

37. The method of claim 36, wherein the method directly induces proliferation of skin cells.

38. The method of claim 37, wherein the skin cells are selected from the group consisting of keratinocytes, fibroblasts, and skin stem cells.

39. A method for treating burned skin, including the step of applying to the burned skin the topical formulation of claim 1.

40. A method of preparing artificial skin, comprising the steps of:
(a) providing an artificial skin substrate;
(b) seeding said artificial skin substrate with skin cells; and
(c) applying the topical formulation of any one of claims claim 1 to said seeded substrate to promote proliferation of said skin cells on said artificial skin substrate.

41. The method of claim 40, wherein the artificial skin is an artificial dermis.

42. The method of claim 41, wherein the artificial dermis is formed using fibroblasts.

43. The method of claim 40, wherein the artificial skin is an artificial epidermis.

44. The method of claim 43, wherein the artificial epidermis is formed using keratinocytes.

45. A method of preparing artificial skin comprising both an artificial dermal layer and an artificial epidermal layer, comprising the steps of:
(a) providing an artificial skin substrate;
(b) seeding said artificial skin substrate with fibroblasts to form an artificial dermal layer;
(c) applying the topical formulation of claim 1 to said fibroblast-seeded substrate to promote proliferation of said fibroblasts in said artificial dermal layer;
(d) seeding said artificial dermal layer with keratinocytes to form an artificial epidermal layer on said dermal layer; and
(e) applying the topical formulation of claim 1 to said keratinocyte-seeded dermal layer to promote proliferation of said keratinocytes in said artificial epidermal layer.

46. An artificial skin composition for use in treating burned skin, where the artificial skin is formed by the methods of claim 40.

47. A method for treating burned skin, including the step of applying to the burned skin the artificial skin substance produced by the methods of claim 40.
